(19) United States
(12) Patent Application Publication
(10) Pub. No.: US 2026/0179770 A1
(43) Pub. Date: Jun. 25, 2026

(54) MACHINE LEARNING MODELING FOR INPATIENT PREDICTION

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Kevin Nicholas, Washington, DC (US); Tiffanny Newman, Bronx, NY (US); Dmitriy Gorenshteyn, Princeton, NJ (US); Melissa S. Pessin, Chicago, IL (US)

(21) Appl. No.: 19/124,666

(22) PCT Filed: Oct. 26, 2023
(86) PCT No.: PCT/US2023/077928

§ 371 Date: Apr. 25, 2025

Related U.S. Application Data
(60) Provisional application No. 63/420,474, filed on Oct. 28, 2022.

(51) Int. Cl. G16H 50/20; G16H 10/60; G16H 50/30; G16H 50/70

(52) U.S. Cl. CPC G16H 50/20; G16H 10/60; G16H 50/30; G16H 50/70

(57) ABSTRACT

Disclosed is an approach that uses artificial intelligence to make predictions regarding patient outcomes, and more specifically, to machine-learning models for inpatient prognosis prediction. A machine-learning classifier may be trained for predicting likelihoods of patients dying a number of days following inpatient admission. A training dataset may comprise, for subjects in a cohort, numerical and categorical values based on a set of tests, as well as demographic or biometric and/or historical values. The machine-learning classifier is trained so as to subsequently output likelihood of patients dying within the number of days following an admission at a healthcare facility.

### CROSS-REFERENCE TO RELATED PATENT APPLICATIONS

This application claims priority to U.S. Provisional Patent Application No. 63/420,474, filed Oct. 28, 2022, the entirety of which is incorporated by reference herein.

### TECHNICAL FIELD

The present technology relates generally to using artificial intelligence to make predictions regarding patient outcomes, and more specifically, to machine-learning models for inpatient prognosis prediction.

### BACKGROUND

When focused on acute issues and when a patient's ultimate prognosis is unclear, it can be difficult to calibrate care plans or know when to facilitate a Goals of Care conversation.

### SUMMARY

In one aspect, various embodiments of the present disclosure relate to a method comprising training a machine-learning classifier for predicting likelihoods of patients dying a first number of days following inpatient admission, wherein training the machine-learning classifier comprises: generating a training dataset using data for subjects in a cohort of study subjects, wherein each subject in the cohort of subjects had a subject admission at a subject healthcare facility, the training dataset comprising, for each subject: (A) numerical values based on a set of laboratory tests run on each subject within a second number of days before or after the subject admission, the numerical values comprising values corresponding to: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to: (i) albumin, (ii) carbon dioxide (CO2), and (iii) calcium corrected for albumin; (C) historical values comprising a count of emergent admissions in a predetermined timeframe preceding the subject admission of each subject; and (D) subject demographic or biometric values corresponding to at least one of body surface area (BSA), body mass index (BMI), height, or weight; applying one or more machine learning techniques to the training dataset such that the machine-learning classifier is configured to receive, as input, numerical values, categorical values, historical values, and one or more demographic or biometric values and provide, as output, likelihood of dying the first number of days following inpatient admission; and providing the machine-learning classifier for use in generating patient-specific likelihoods of dying the first number of days following respective inpatient admissions.

In various embodiments, mortality at the first number of days following the subject admission is known for each subject, and wherein the one or more machine learning techniques comprises one or more supervised machine learning techniques.

In various embodiments, the one or more machine learning techniques comprises a regression analysis model. In various embodiments, the regression analysis model is based on Least Absolute Shrinkage and Selection Operator (LASSO).

In various embodiments, the first number of days is between 30 and 90. In various embodiments, the first number of days is 45.

In various embodiments, each subject in the cohort of subjects had a cancer. In various embodiments, each subject in the cohort had a non-cancerous disease.

In various embodiments, each subject in the cohort of subjects had a solid tumor. In various embodiments, each subject in the cohort of subjects had a hematological malignancy.

In various embodiments, the method further comprises predicting, using the machine-learning classifier, a likelihood of a patient dying the first number of days following an admission of the patient at a healthcare facility.

In various embodiments, using the machine-learning model comprises selecting a set of features for predicting the likelihood a non-zero coefficient, wherein the set of features are selected based on predictive coefficients of features.

In another aspect, various embodiments of the present disclosure relate to a method comprising training a machine-learning classifier for predicting likelihoods of patients dying a first number of days following inpatient admission, wherein training the machine-learning classifier comprises: generating a training dataset using data for subjects in a cohort of study subjects, wherein mortality at the first number of days following the subject admission is known for each subject, and wherein each subject in the cohort of subjects had a subject admission at a subject healthcare facility, the training dataset comprising, for each subject: (A) numerical values based on a set of laboratory tests run on each subject within a second number of days before or after the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject demographic or biometric values corresponding to at least one of body surface area (BSA), body mass index (BMI), height, or weight; applying one or more supervised machine learning techniques to the training dataset such that the machine-learning classifier is configured to receive, as input, numerical values, categorical values, historical values, and one or more demographic or biometric values and provide, as output, likelihood of dying the first number of days following inpatient admission; and providing the classifier for use in generating patient-specific likelihoods of dying the first number of days following respective inpatient admissions.

In various embodiments, the one or more machine learning techniques comprises a regression analysis model. In various embodiments, the regression analysis model is based on Least Absolute Shrinkage and Selection Operator (LASSO).

In various embodiments, the first number of days is between 30 and 90.

In various embodiments, each subject in the cohort of subjects had a solid tumor. In various embodiments, each subject in the cohort of subjects had a hematological malignancy.

In various embodiments, the method further comprises using the machine-learning classifier to predict a likelihood of dying the first number of days following an admission of the patient at a healthcare facility.

In yet another aspect, various embodiments of the present disclosure relate to a method comprising: receiving, by a first computing device, for a patient admitted to a healthcare facility, health data comprising: (A) numerical values based on a set of laboratory tests run on the patient, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject physical characteristic values corresponding to at least one of body surface area (BSA), body mass index (BMI), height, or weight; inputting, by the first computing device, the health data to a machine-learning classifier to generate a likelihood that the patient will die a number of days following admission to the healthcare facility, the machine learning classifier having been trained, by the first computing device or a second computing device, by: generating a training dataset using data for subjects in a cohort of study subjects, wherein each subject in the cohort of subjects had a subject admission at a subject healthcare facility, the training dataset comprising, for each subject: (A) numerical values based on a set of laboratory tests run on each subject following the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject physical characteristic values corresponding to at least one of body surface area (BSA), body mass index (BMI), height, or weight; and applying one or more machine learning techniques to the training dataset to obtain the classifier such that the configured to receive, as input, numerical values, categorical values, historical values, and one or more demographic or biometric values and provide, as output, likelihood of dying the number of days following inpatient admission; and providing, by the first computing device, the likelihood generated by the machine-learning classifier to a healthcare provider for use in care of the patient.

In various embodiments, mortality at the number of days following the subject admission is known for each subject, and wherein the one or more machine learning techniques comprises one or more supervised machine learning techniques.

In various embodiments, the one or more machine learning techniques comprises a LASSO regression model.

In various embodiments, the number of days is between 40 and 50.

In yet other aspects, various embodiments of the present disclosure relate to a computing system (which may be, or may comprise, one or more computing devices) comprising one or more processors configured to implement any of the methods disclosed in the present disclosure.

In yet other aspects, various embodiments of the present disclosure relate to a non-transitory computer-readable storage medium with instructions configured to cause one or more processors of a computing system (which may be, or may comprise, one or more computing devices) to implement any of the methods disclosed in the present disclosure.

In yet other aspects, various embodiments of the disclosure relate to processes performed using devices and/or systems disclosed herein.

### BRIEF DESCRIPTION OF THE DRAWINGS

FIG. 1 depicts an example system for implementing the disclosed approach, according to various potential embodiments.

FIG. 2 depicts example modeling and prediction processes, according to various potential embodiments.

FIG. 3 shows a simplified block diagram of a representative server system and client computer system usable to implement certain embodiments of the present disclosure.

FIG. 4 represents an example production workflow, according to potential embodiments of the disclosed approach.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, devices, or systems, each of which can, of course, vary. It is also to be understood that the terminology used herein is for the purpose of describing particular embodiments only, and is not intended to be limiting.

Clinicians struggle with having appropriate end-of-life care discussions with hospitalized patients at suitable times. Often such discussions are only considered when it is too late, such as when a patient is decompensating, struggling, or in urgent need of intervention. Identifying patients ad hoc allows for the introduction of explicit and implicit biases, meaning clinicians are only correct some of the time regarding who and when they have difficult but important serious illness discussions.

Some patients admitted are sick, and it is unclear what their trajectory will be. In various embodiments, a tool that incorporates, for example, key laboratory results will help clinicians better assess the chance of a patient passing away within, for example, 45 days of admission, thereby guiding decisions about these patients' care plans. The inpatient admissions may be, for example, non-surgical admissions to the department of medicine.

In various embodiments, regression modeling, and in particular a regression analysis model referred to as LASSO (Least Absolute Shrinkage and Selection Operator), allows for the use of select features to generate useful prognoses that enhance patient care. For example, the LASSO model is highly generalizable and easily interpretable. The model's coefficients give users (e.g., clinicians) some indication as to why the model is predicting the patients are at high risk. It is submitted that if clinicians are able to interpret details about the model, that ability will foster clinical adoption and clinical trust in the predictions from the model.

In alternative embodiments, an XGBoost version of the model may be employed. XGBoost is based on distributed gradient-boosted decision trees (GBDT), a decision tree ensemble learning algorithm similar to random forest, for classification and regression. It has been shown that XGBoost may perform better in certain circumstances, but the model may be less interpretable, resulting in a tradeoff. In example implementations, an example XGBoost model's performance on a validation dataset was shown to be 0.84. LASSO and other machine learning (ML) models (used alone or in combination) as used here are highly complex, with an iterative nature that seeks the best combinations of hyperparameters to tune the ML models to find the best model.

In an example clinical workflow, when a patient is admitted to a hospital, the day shift clinicians are primarily responsible for the patient's care. Every morning at 7:00 am, for example, the day shift clinicians take over from the night shift. At 7:00 am, the day shift Advanced Practice Providers (APPs) and nurses review the patients' electronic medical record (EMR)/electronic health record (EHR) to brief an attending doctor about the patients' status before “rounding” (i.e., before patient and clinician bedside meetings). During the “pre-rounding” briefing session, clinicians develop the patient's care plan (e.g., what tests or procedures to order). Typically, the clinicians are trying to manage the patients' acute needs. In addressing the patients' critical needs, a long-term needs plan may be overlooked.

The disclosed model brings attention to long-term needs, especially those regarding end-of-life care. Often, such topics are considered too late to be effective. The preferred time to alert clinical teams to a patient's risk of death is during this “pre-rounding” briefing session. For this reason, a pipeline to produce predictions may be initiated at 7:00 am in this scenario, and predictions may be pushed to the patient's EMR, allowing all clinicians to view through a clinical information system (CIS).

The predictions may then prompt clinicians to reach out to, for example, the patients' primary oncologist to facilitate end-of-life preparations or prompt the hospital staff to re-evaluate high-risk patients who seemed not at risk. In most instances, the high-risk predictions would be expected to trigger clinicians to provide additional care and seek support from the appropriate consulting services.

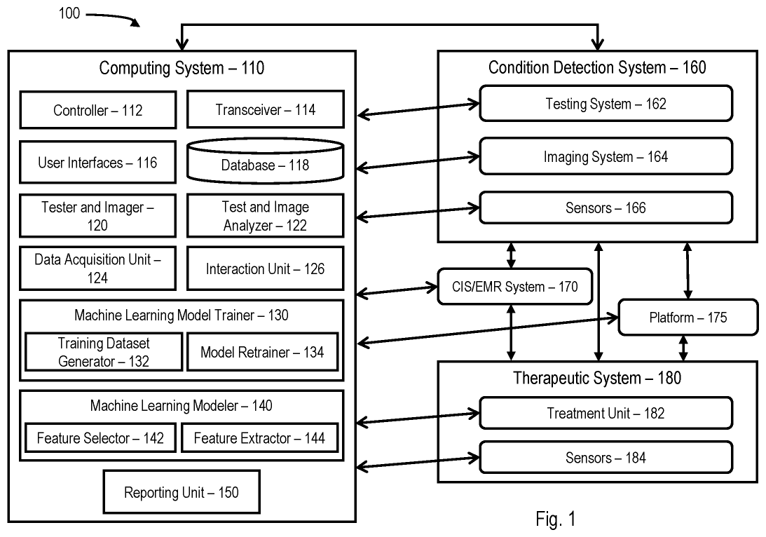

Referring to FIG. 1, in various embodiments, a system 100 may include a computing system 110 (which may be or may include one or more computing devices, co-located or remote to each other), a condition detection system 160, an Clinical Information System (CIS) 170 (used interchangeably with electronic medical record (EMR) system 170), a platform 175, and a therapeutic system 180. The computing system 110 (one or more computing devices) may be used to control and/or exchange signals and/or data with condition detection system 160, EMR system 170, platform 175, and/or therapeutic system 180, directly or via another component of system 100. In certain embodiments, computing system 110 may be used to control and/or exchange data or other signals with condition detection system 160, EMR system 170, platform 175, and/or therapeutic system 180. The computing system 110 may include one or more processors and one or more volatile and non-volatile memories for storing computing code and data that are captured, acquired, recorded, and/or generated.

The computing system 110 may include a controller 112 that is configured to exchange control signals with condition detection system 160, EMR system 170, platform 175, therapeutic system 180, and/or any components thereof, allowing the computing system 110 to be used to control, for example, acquisition of patient data such as test results, capture of images, acquisition of signals by sensors, positioning or repositioning of subjects and patients, recording or obtaining other subject or patient information, and applying therapies.

A transceiver 114 allows the computing system 110 to exchange readings, control commands, and/or other data, wirelessly or via wires, directly or via networking protocols, with condition detection system 160, EMR system 170, platform 175, and/or therapeutic system 180, or components thereof. One or more user interfaces 116 allow the computing device 110 to receive user inputs (e.g., via a keyboard, touchscreen, microphone, camera, etc.) and provide outputs (e.g., via a display screen, audio speakers, etc.) with users. The computing device 110 may additionally include one or more databases 118 for storing, for example, data acquired from one or more systems or devices, signals acquired via one or more sensors, biomarker signatures, etc. In some implementations, database 118 (or portions thereof) may alternatively or additionally be part of another computing device that is co-located or remote (e.g., via “cloud computing”) and in communication with computing device 110, condition detection system 160, EMR system 170, platform 175, and/or therapeutic system 180 or components thereof.

Condition detection system 160 may include a testing system 162, which may be or may include, for example, any system or device that is involved in, for example, analyzing samples, recording patient data, and/or obtaining laboratory or other test results. An imaging system 164 may be any system or device used to capture imaging data, such as a magnetic resonance imaging (MRI) scanner, a positron emission tomography (PET) scanner, a single photon emission computed tomography (SPECT) scanner, a computed tomography (CT) scanner, a fluoroscopy scanner, and/or other imaging devices and/or sensors). Sensors 166 may detect, for example, a position or motion of a patient, organs, tissues, physiological readings such as lung capacity or heart activity/signals, or other states and/or conditions of the patient.

Therapeutic system 180 may include a treatment unit 180, which may be or may include, for example, a radiation source for external beam therapy (e.g., orthovoltage x-ray machines, Cobalt-60 machines, linear accelerators, proton beam machines, neutron beam machines, etc.) and/or one or more other treatment devices. Sensors 184 may be used by therapeutic system 180 to evaluate and guide a treatment (e.g., by detecting level of emitted radiation, a condition or state of the patient, or other states or conditions). In various implementations, components of system 100 may be rearranged or integrated in other configurations. For example, computing system 110 (or components thereof) may be integrated with one or more of the condition detection system 160, therapeutic system 180, and/or components thereof. The condition detection system 160, therapeutic system 180, and/or components thereof may be directed to a platform 175 on which a patient, subject, or sample can be situated (so as to test a sample, image a subject, apply a treatment or therapy to the subject, detect activity and/or motion of the subject in a stress test, etc.). In various embodiments, the platform 175 may be movable (e.g., using any combination of motors, magnets, etc.) to allow for positioning and repositioning of samples and subjects (such as micro-adjustments to compensate for motion of a subject or patient or to position the patient for scans of different regions of interest). The platform 175 may include its own sensors to detect a condition or state of the sample, patient, and/or subject.

The computing system 110 may include a tester and imager 120 configured to direct, for example, laboratory tests, image capture, and acquisition of test and/or imaging data. Tester and imager 120 may include an image generator that may convert or transform raw imaging data from condition detection system 160 into usable medical images or into another form to be analyzed. Computing system 110 may include a test and image analyzer 122 configured to, for example, analyze raw test results to generate relevant values, identify features in images or imaging data, or otherwise make use of tests or testing data, and/or images or imaging data.

A data acquisition unit 124 may retrieve, acquire, or otherwise obtain various data to be used to, for example, train models using data on subjects, or apply trained models using data on patients. The data acquisition unit 124 may, for example, obtain test data that stored in EMR system 170. An interaction unit 126 may interact (e.g., via user interfaces 116) with users (e.g., patients and/or healthcare providers) to obtain information needed for a model or to provide information to users. In certain embodiments, the data acquisition unit 124 may obtain data from users via interaction unit 126.

A machine learning model trainer 130 (“ML model trainer”) may be configured to train machine learning models used herein, as further discussed below. The ML model trainer may include a training dataset generator that processes various data to generate one or more training datasets to be used, for example, to train the models discussed herein. In certain embodiments, as more data and outcomes become available, a model retrainer 134 may further train a previously-trained model to improve or otherwise update or revise models. The ML model trainer may apply various machine learning techniques to the training datasets, such as LASSO, XGBoost, random forest, and/or other machine learning techniques. A machine learning modeler 140 (“ML modeler”) may be configured to apply trained machine learning models to particular patient data. A feature selector 142 may be configured to select which features are to be fed to a model to obtain a prognosis prediction (e.g., a likelihood of dying in 45 days). The feature selector 142 may select features based on, for example, which features were previously employed to train the ML model(s) to be used, the parameters of the model(s), and/or how influential the features were in more predicting outcomes (e.g., feature coefficients). The feature selector 142 may additionally or alternatively select features based on what data (e.g., test results) are available for the patient. For example, if data corresponding to one feature in a “tier 1” feature set (e.g., the “top five” most influential features with the five highest coefficients) is not available for the patient, two features in a “tier 2” feature set (e.g., features with the 6th to 10th highest coefficients) may be selected instead. A feature extractor 144 may obtain values for selected features from various data sources. A reporting unit 150 may generate reports that include, for example, model outputs (e.g., likelihood of death) along with information on the basis for a prognosis, so as to identify for clinicians the factors most impactful on low or high likelihoods or other prognoses.

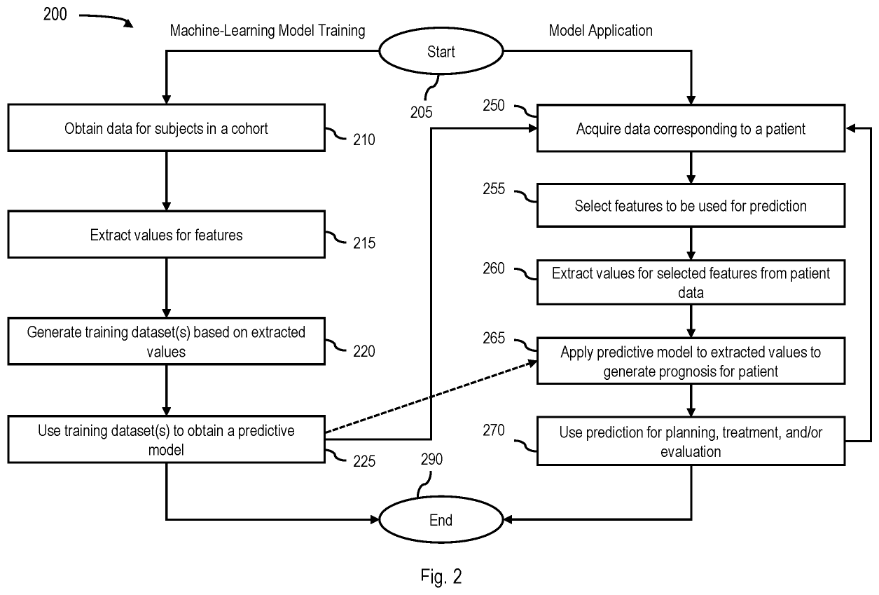

Referring to FIG. 2, an example process 200 is illustrated, according to various potential embodiments. Various elements of process 200 may be implemented by or via system 100 or components thereof. Process 200 may begin (205) with model training (on the left side of FIG. 2), which may be implemented by or via computing system 110, if a model is not already available (e.g., in database 118), or if additional models are to be generated or updated through training or retraining with new training data. Alternatively, process 200 may begin with use (application) of a model (on the right side of FIG. 2) for a patient if a trained model is already available. Prognosis prediction may be implemented by or via computing system 110 if a suitable trained model is available. In various embodiments, process 200 may comprise both model training (e.g., steps 210-225) followed by prognosis prediction (e.g., steps 250-270).

At 210, health data pertaining to subjects may be obtained. This may be obtained by or via, for example, condition detection system 160 and/or EMR system 170 for a cohort of, for example, 10,000, 11,000, 12,000, 13,000, or 14,000 subjects across several diseases. This may represent, for example, two years of admissions. The user should have enough data, across multiple diseases to account for differences in those diseases and build a generalizable model. Test or imaging data may be transformed, converted, or otherwise analyzed by test and image analyzer 122. Step 215 involves extracting, from (or based on) the health data, feature values corresponding to the subjects in the cohort.

At 220, one or more datasets (e.g., training datasets) may be generated using the extracted feature values corresponding health data of the subjects. This may be performed, for example, by or via ML model trainer 130, or more specifically, training dataset generator 132. At 225, the one or more datasets may be used to train a model for prognosis predictions. The model may be stored (e.g., in database 118) for subsequent use. Process 200 may end (290), or may proceed to step 250 for use in prognosis prediction. (As represented by the dotted line from step 225 to step 265, the model may subsequently be used to generate and use prognosis prediction.)

At 250, health data of a patient may be obtained and analyzed (e.g., by or via condition detection system 160 and/or EMR system 170). Test data may correspond to various laboratory tests performed on samples of the patient. At 255, a set of features may be selected (e.g., by or via feature selector 142), and at 260, values for the features may be extracted from the health data of the patient (e.g., by or via feature extractor 144).

At 265, feature values extracted from patient health data may be input to a predictive model (e.g., a machine learning classifier) to predict patient prognosis. At 270, the predicted prognosis and the factors underlying the prognosis may be used in caring for the patient. For example, predicted prognosis may be used for planning for a potential outcome and/or identifying potentially preventative care. Additionally or alternatively, predicted prognosis can be used in evaluation of a patient's health and changes or trends therein, such as whether the patient is deteriorating or improving as indicated by changes in prognoses over time from the ML modeler as new tests are run or otherwise new health data becomes available.

Process 200 may end (290), or return to step 250 (e.g., after running another test or administering a treatment) for subsequent planning based on a change in a condition of the patient.

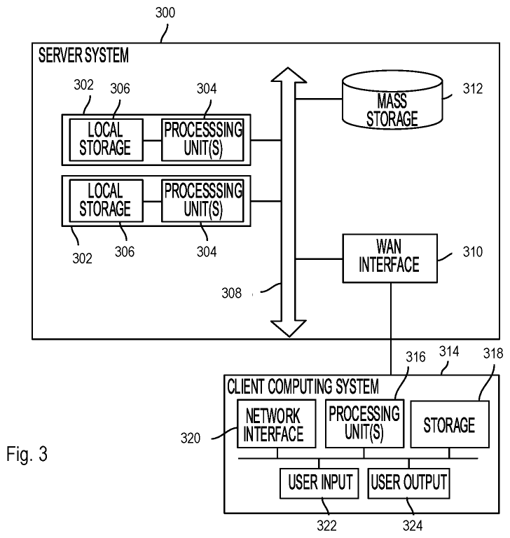

Various operations described herein can be implemented on computer systems having various configurations. FIG. 3 shows a simplified block diagram of a representative server system 300 (e.g., computing system 110) and client computer system 314 (e.g., computing system 110, condition detection system 160, EMR system 170, and/or therapeutic system 180) usable to implement various embodiments of the present disclosure. In various embodiments, server system 300 or similar systems can implement services or servers described herein or portions thereof. Client computer system 314 or similar systems can implement clients described herein.

Server system 300 can have a modular design that incorporates a number of modules 302 (e.g., blades in a blade server embodiment); while two modules 302 are shown, any number can be provided. Each module 302 can include processing unit(s) 304 and local storage 306.

Processing unit(s) 304 can include a single processor, which can have one or more cores, or multiple processors. In some embodiments, processing unit(s) 304 can include a general-purpose primary processor as well as one or more special-purpose co-processors such as graphics processors, digital signal processors, or the like. In some embodiments, some or all processing units 304 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 304 can execute instructions stored in local storage 306. Any type of processors in any combination can be included in processing unit(s) 304.

Local storage 306 can include volatile storage media (e.g., conventional DRAM, SRAM, SDRAM, or the like) and/or non-volatile storage media (e.g., magnetic or optical disk, flash memory, or the like). Storage media incorporated in local storage 306 can be fixed, removable or upgradeable as desired. Local storage 306 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 304 need at runtime. The ROM can store static data and instructions that are needed by processing unit(s) 304. The permanent storage device can be a non-volatile read-and-write memory device that can store instructions and data even when module 302 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 306 can store one or more software programs to be executed by processing unit(s) 304, such as an operating system and/or programs implementing various server functions or any system or device described herein.

“Software” refers generally to sequences of instructions that, when executed by processing unit(s) 304 cause server system 300 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 304. Software can be implemented as a single program or a collection of separate programs or program modules that interact as desired. From local storage 306 (or non-local storage described below), processing unit(s) 304 can retrieve program instructions to execute and data to process in order to execute various operations described above.

In some server systems 300, multiple modules 302 can be interconnected via a bus or other interconnect 308, forming a local area network that supports communication between modules 302 and other components of server system 300. Interconnect 308 can be implemented using various technologies including server racks, hubs, routers, etc.

A wide area network (WAN) interface 310 can provide data communication capability between the local area network (interconnect 308) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 306 is intended to provide working memory for processing unit(s) 304, providing fast access to programs and/or data to be processed while reducing traffic on interconnect 308. Storage for larger quantities of data can be provided on the local area network by one or more mass storage subsystems 312 that can be connected to interconnect 308. Mass storage subsystem 312 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 312. In some embodiments, additional data storage resources may be accessible via WAN interface 310 (potentially with increased latency).

Server system 300 can operate in response to requests received via WAN interface 310. For example, one of modules 302 can implement a supervisory function and assign discrete tasks to other modules 302 in response to received requests. Conventional work allocation techniques can be used. As requests are processed, results can be returned to the requester via WAN interface 310. Such operation can generally be automated. Further, in some embodiments, WAN interface 310 can connect multiple server systems 300 to each other, providing scalable systems capable of managing high volumes of activity. Conventional or other techniques for managing server systems and server farms (collections of server systems that cooperate) can be used, including dynamic resource allocation and reallocation.

Server system 300 can interact with various user-owned or user-operated devices via a wide-area network such as the Internet. An example of a user-operated device is shown in FIG. 3 as client computing system 314. Client computing system 314 can be implemented, for example, as a consumer device such as a smartphone, other mobile phone, tablet computer, wearable computing device (e.g., smart watch, eyeglasses), desktop computer, laptop computer, and so on.

Client computing system 314 can communicate via WAN interface 310. Client computing system 314 can include conventional computer components such as processing unit(s) 316, storage device 318, network interface 320, user input device 322, and user output device 324. Client computing system 314 can be a computing device implemented in a variety of form factors, such as a desktop computer, laptop computer, tablet computer, smartphone, other mobile computing device, wearable computing device, or the like.

Processor 316 and storage device 318 can be similar to processing unit(s) 304 and local storage 306 described above. Suitable devices can be selected based on the demands to be placed on client computing system 314; for example, client computing system 314 can be implemented as a “thin” client with limited processing capability or as a high-powered computing device. Client computing system 314 can be provisioned with program code executable by processing unit(s) 316 to enable various interactions with server system 300 of a message management service such as accessing messages, performing actions on messages, and other interactions described above. Some client computing systems 314 can also interact with a messaging service independently of the message management service.

Network interface 320 can provide a connection to a wide area network (e.g., the Internet) to which WAN interface 310 of server system 300 is also connected. In various embodiments, network interface 320 can include a wired interface (e.g., Ethernet) and/or a wireless interface implementing various RF data communication standards such as Wi-Fi, Bluetooth, or cellular data network standards (e.g., 3G, 4G, 5G, LTE, etc.).

User input device 322 can include any device (or devices) via which a user can provide signals to client computing system 314; client computing system 314 can interpret the signals as indicative of particular user requests or information. In various embodiments, user input device 322 can include any or all of a keyboard, touch pad, touch screen, mouse or other pointing device, scroll wheel, click wheel, dial, button, switch, keypad, microphone, and so on.

User output device 324 can include any device via which client computing system 314 can provide information to a user. For example, user output device 324 can include a display to display images generated by or delivered to client computing system 314. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 324 can be provided in addition to or instead of a display. Examples include indicator lights, speakers, tactile “display” devices, printers, and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 304 and 316 can provide various functionality for server system 300 and client computing system 314, including any of the functionality described herein as being performed by a server or client, or other functionality associated with message management services.

It will be appreciated that server system 300 and client computing system 314 are illustrative and that variations and modifications are possible. Computer systems used in connection with embodiments of the present disclosure can have other capabilities not specifically described here. Further, while server system 300 and client computing system 314 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. For instance, although specific examples of rules (including triggering conditions and/or resulting actions) and processes for generating suggested rules are described, other rules and processes can be implemented. Embodiments of the disclosure can be realized using a variety of computer systems and communication technologies including but not limited to specific examples described herein.

Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

The present technology is further illustrated by the following Examples, which should not be construed as limiting in any way.

### EXAMPLES

### Modeling Framework

In various embodiments, the model primarily relies on patient numerical laboratory results from tests included in the comprehensive metabolic and the complete blood count with differential panels and certain other laboratory tests, such as prothrombin time (PT), activated partial thromboplastin time (APTT), magnesium, phosphorus, and lactate dehydrogenase. Additionally, in various embodiments, the model uses oncology-specific reference ranges for certain laboratory tests with non-linear relationships (e.g., albumin, calcium, carbon dioxide, chloride, and potassium). In various embodiments, laboratory tests evaluated are limited to the last result available anywhere in the window of one day before through to the time of the prediction (e.g., 7:00 am).

In addition to laboratory tests, in various embodiments the model also uses the patient's age, gender, body surface area (BSA), height, weight, body mass index (BMI), 30-day change in BMI, history of previous urgent admissions, and the admitting service (e.g., the solid tumor admitting service or hematological admitting service). In certain embodiments, non-laboratory test results (such as physiological readings from the heart, lungs, or brain during a certain activity or state) could alternatively or additionally be used.

A set of sample feature data for is provided in Table 1:

TABLE 1
Sample Feature Data (Model Inputs), where “Cat” refers to a categorical
variable and “Num” refers to a numerical variable.

| Feature Data | Value | Type |
|---|---|---|
| gs_split | TRAIN | Cat |
| patient.age | 74 | Num |
| patient.GENDER | MALE | Cat |
| patient.height | 166 | Num |
| patient.weight | 69.6 | Num |
| patient.bsa | 1.8 | Num |
| patient.bmi | 24.9 | Num |
| patient.bmi_weight_group | UNKNOWN | Cat |
| patient.bmi_change_30d | 0 | Num |
| admit.SERVICE_ADMIT | Urologic Med | Cat |
| admit.COUNT_EMG_DISCHARGES | 0 | Num |
| labs_num.Abs_Baso | 0 | Num |
| labs_num.Abs_Eos | 0 | Num |
| labs_num.Abs_Lymph | 0.9 | Num |
| labs_num.Abs_Mono | 0.7 | Num |
| labs_num.Abs_Neut | 2.4 | Num |
| labs_num.Absolute_Immature_Granulocyte | 0 | Num |
| labs_num.Alanine_Aminotransferase_ALT | 8 | Num |
| labs_num.Albumin | 3.3 | Num |
| labs_num.Alkaline_Phosphatase_ALK | 337 | Num |
| labs_num.Anion_Gap_Calc | 16 | Num |
| labs_num.Aspartate_Aminotransferase_AST | 43 | Num |
| labs_num.Baso | 0 | Num |
| labs_num.Bilirubin_Direct | 0.3 | Num |
| labs_num.Bilirubin_Total | 0.9 | Num |
| labs_num.BUN | 51 | Num |
| labs_num.Calcium | 9 | Num |
| labs_num.Carbon_Dioxide_CO2 | 12 | Num |
| labs_num.Chloride | 107 | Num |
| labs_num.Creatinine | 1.5 | Num |
| labs_num.Eos | 0.2 | Num |
| labs_num.Glucose | 268 | Num |
| labs_num.HCT | 28.2 | Num |
| labs_num.HGB | 9.3 | Num |
| labs_num.Immature_Granulocyte | 1.2 | Num |
| labs_num.Lymph | 23 | Num |
| labs_num.Magnesium | 2.2 | Num |
| labs_num.MCH | 30.5 | Num |
| labs_num.MCV | 93 | Num |
| labs_num.Mean_Corpuscular_Hemoglobin_Conc_MCHC | 33 | Num |
| labs_num.Mono | 17.3 | Num |
| labs_num.Neutrophil | 58.3 | Num |
| labs_num.Platelets | 195 | Num |
| labs_num.Potassium | 4.5 | Num |
| labs_num.Protein_Total_Plasma | 7.2 | Num |
| labs_num.RBC | 3.05 | Num |
| labs_num.RDW | 17.5 | Num |
| labs_num.Sodium | 135 | Num |
| labs_num.WBC | 4 | Num |
| labs_cat_rev.Albumin | Very_Low | Cat |
| labs_cat_rev.Calcium | Normal | Cat |
| labs_cat_rev.Calcium_Corrected | Normal | Cat |
| labs_cat_rev.Carbon_Dioxide_CO2 | OTHER | Cat |
| labs_cat_rev.Chloride | Normal | Cat |
| labs_cat_rev.Potassium | Normal | Cat |
| labs_cat_rev.Sodium | Normal | Cat |
| gs_label | FALSE | Cat |

Table 2 provides a set of features as well as their predictive coefficients with respect to what is being predicted: death within 45 days of admission. Positive coefficients indicate that a feature increases risk as its magnitude increases. Negative coefficients exhibit the opposite dynamic: for features with negative coefficients, risk decreases as their values increases. Therefore, in this situation, features with negative coefficients can be described as “protective” in the sense that they “protect” against the outcome being predicted (death within 45 days of admission to a healthcare facility). For example, for albumin, the higher the value of the laboratory test result, the more protective because that value is multiplied by a negative coefficient. In contrast, lower albumin results result in reduced protection. Consequently, the magnitude of the coefficients indicate the impact on risk, while the sign indicates whether they increase risk (positive values) or decrease risk (negative values).

TABLE 2
Sample Coefficients for Sample Features

| No. | Type | Feature | Coefficient |
|---|---|---|---|
| 1 | Numeric Lab Test Value | Creatinine | −0.772 |
| 2 | Categorical Lab Test Value | Calcium Corrected | 0.614 |
| 3 | Categorical Lab Test Value | Albumin | −0.452 |
| 4 | Numeric Lab Test Value | Anion Gap Calculation | 0.371 |
| 5 | Numeric Lab Test Value | Lymphocytes | −0.364 |
| 6 | Numeric Lab Test Value | Mean Corpuscular Volume MCV | 0.352 |
| 7 | Numeric Lab Test Value | Mean Corpuscular Hemoglobin MCH | −0.347 |
| 8 | Numeric Lab Test Value | Red blood cell Distribution Width RDW | 0.31 |
| 9 | Categorical Lab Test | Carbon Dioxide CO2 | 0.298 |
| 10 | Numeric Lab Test Value | BUN | 0.277 |
| 11 | Numeric Lab Test Value | Aspartate Aminotransferase AST | 0.273 |
| 12 | Numeric Lab Test Value | Potassium | 0.238 |
| 13 | Numeric Lab Test Value | Alanine Aminotransferase ALT | −0.21 |
| 14 | Demographics & Biometric Data | Body Surface Area BSA | −0.205 |
| 15 | Numeric Lab Test Value | Eosinophils | 0.189 |
| 16 | Numeric Lab Test Value | Neutrophil | 0.175 |
| 17 | Feature of the Admission | Admitting Service | 0.172 |
| | (Historical) | | |
| 18 | Categorical Lab Test Value | Sodium | 0.162 |
| 19 | Patient Demographics & | Height | 0.149 |
| | Biometric Data | | |
| 20 | Patient Demographics & | BMI Weight Group | 0.145 |
| | Biometric Data | | |
| 21 | Numeric Lab Test Value | Absolute Eosinophils | 0.14 |
| 22 | Feature of the Admission | Count of Emergent Admissions in the Last | 0.139 |
| | (Historical) | 60 Days | |
| 23 | Numeric Lab Test Value | Glucose | −0.135 |
| 24 | Numeric Lab Test Value | Platelets | −0.132 |
| 25 | Numeric Lab Test Value | Protein Total Plasma | −0.13 |
| 26 | Numeric Lab Test Value | Hematocrit HCT | 0.129 |
| 27 | Numeric Lab Test Value | Immature Granulocyte | 0.096 |
| 28 | Numeric Lab Test Value | Mean Corpuscular Hemoglobin Conc MCHC | 0.085 |
| 29 | Numeric Lab Test Value | Calcium | 0.082 |
| 30 | Numeric Lab Test Value | Red Blood Cell (RBC) count | −0.082 |
| 31 | Patient Demographics & | BMI weight group weight | −0.076 |
| | Biometric Data | | |
| 32 | Numeric Lab Test Value | Absolute Monocytes | 0.075 |
| 33 | Patient Demographics & | BMI | 0.072 |
| | Biometric Data | | |
| 34 | Numeric Lab Test Value | Bilirubin Total | 0.068 |
| 35 | Numeric Lab Test Value | Absolute Immature Granulocyte | −0.066 |
| 36 | Demographics & Biometric Data | Gender | 0.063 |
| 37 | Numeric Lab Test Value | Absolute Neutrophil | 0.06 |
| 38 | Numeric Lab Test Value | Basophils | −0.06 |
| 39 | Numeric Lab Test Value | Monocytes | −0.052 |
| 40 | Patient Demographics & | Age | 0.051 |
| | Biometric Data | | |
| 41 | Numeric Lab Test Value | Magnesium | 0.046 |
| 42 | Numeric Lab Test Value | Absolute Basophil Count | 0.034 |
| 43 | Numeric Lab Test Value | White Blood Count WBC | −0.033 |
| 44 | Patient Demographics & | Weight | −0.032 |
| | Biometric Data | | |
| 45 | Numeric Lab Test Value | Absolute Lyphomcytes | 0.015 |
| 46 | Numeric Lab Test Value | Bilirubin Direct | −0.007 |
| 47 | Categorical Lab Test Value | Chloride | −0.007 |
| 48 | Numeric Lab Test Value | Hemoglobin HGB | −0.002 |
| 49 | Patient Demographics & | BMI change in past 30 days | 0 |
| | Biometric Data | | |

Performance: The time-based approach to splitting the Train, Validate, and Test data sets was used because medicine evolves over reasonably short periods. New treatments become available, and practice guidelines change. Applicant sought to test the model with the latest data, not randomly selected data, to get a more accurate reflection of how the model performs in a live setting.

The Test dataset is a separate set of data used to test the model after completing the training. It provides an unbiased final model performance metric in terms of accuracy, precision, etc. To put it simply, it answers the question of “How well does the model perform?” The Test dataset has solid tumor admissions from July 2021 to December 2021.

Tables 3, 4, and 5 provide performance data with respect to the Train, Validate, and Test Datasets. Train refers to the set of data that is used to train and make the model learn the hidden features and patterns in the data. The Train dataset has solid tumor admissions from 2017 to 2020. Validate is separate from the training set, that is used to validate model performance during training. This validation process gives information that helps tune the model's hyperparameters and configurations accordingly. The Validation dataset has solid tumor admissions from January 2021 to June 2021. The Test dataset is a separate set of data used to test the model after completing the training. It provides an unbiased final model performance metric in terms of accuracy, precision, etc. To put it simply, it answers the question of “How well does the model perform?” The Test dataset has solid tumor admissions from July 2021 to December 2021.

TABLE 3
Original Model Performance

| Data Set | Count of Admissions | AUC |
|---|---|---|
| TRAIN | 29,840 | 0.82* |
| VALIDATE | 3,618 | 0.82* |
| TEST | 3,718 | 0.83* |

*Model Performance is evaluated using the Test AUC

Table 4 provides data on model performance using 20 features with the highest coefficient magnitudes. As can be seen, the model performed comparably with this more-limited number of features.

TABLE 4
Model Performance - Using Only Top 20 Features

| Data Set | Count of Admissions | AUC |
|---|---|---|
| TRAIN | 29,840 | 0.81* |
| VALIDATE | 3,618 | 0.82* |
| TEST | 3,718 | 0.82* |

*Model Performance is evaluated using the Test AUC

Table 5 provides data on model performance using 20 numeric features with the highest coefficient magnitudes. As can be seen, the model performed comparably with this subset of features.

TABLE 5
Model Performance - Using Only Top 20 Numeric Features

| Data Set | Count of Admissions | AUC |
|---|---|---|
| TRAIN | 29,840 | 0.81* |
| VALIDATE | 3,618 | 0.81* |
| TEST | 3,718 | 0.82* |

*Model Performance is evaluated using the Test AUC

It is noted that although the models discussed in the above Examples section use data on solid tumors, the disclosed approach is also applicable to non-solid cancers (e.g., hematological malignancies) as well as non-cancerous diseases. It is also noted that the features employed by various embodiments of the disclosed models are not limited to the above features, and may additionally employ one or more metrics related to, for example, vitals and information on previous hospice care that was provided or recommended.

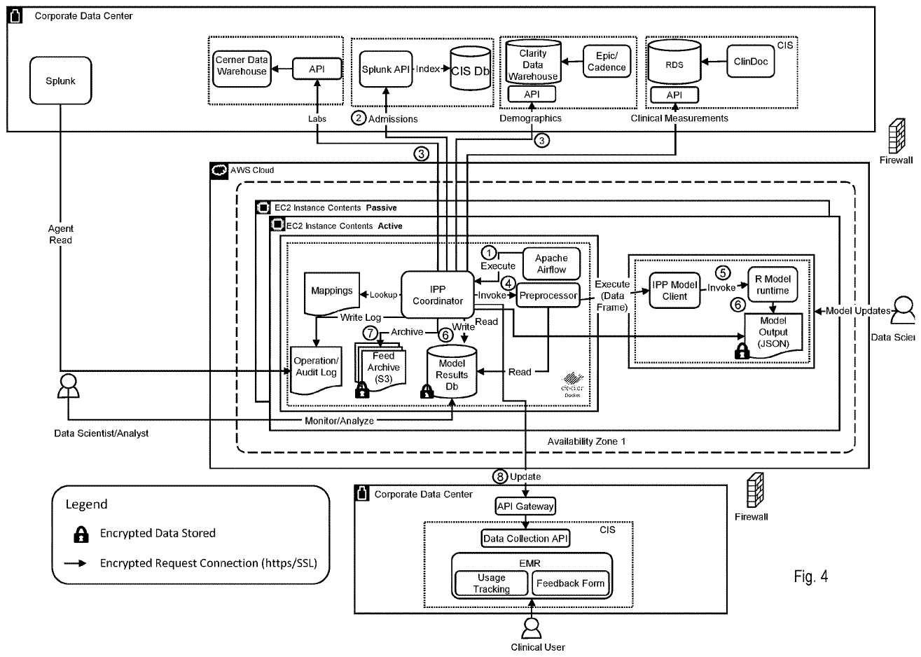

Turning to FIG. 4, an example production workflow will now be discussed. At 7:00 am each morning, a pipeline is initiated via Apache Airflow and Amazon Web Services (AWS). The depicted pipeline illustrates three major points highlighted here. First, for every new patient admitted to the hospital floor within the last day, this pipeline pulls that patient's data and stores it in a PostgreSQL (also referred to as PostGres) database on AWS (steps 1-4). Second, the pipeline initiated Docker tasks that will process all the data and prepare a table of features the model will use to make a prediction. The Docker tasks also execute the model on said data and produce a prediction of High- or Low-risk of death within 45 days, these data are also saved in the PostGres database on AWS (steps 4-6). Finally, this prediction is pushed to the patient EMR/CIS Application via application programming interface (API) for users to consume. In this example scenario, it takes about one to two minutes to produce predictions. Steps 1-7 are as follows, according to various embodiments:
Step 1: On a daily (or other periodic or regular basis) basis, Apache Airflow schedules a call to invoke IPP Coordinator;
Step 2: IPP Coordinator calls Splunk API to retrieve and identify new admissions at healthcare facility;
Step 3: For each new admitted patient, IPP Coordinator obtain laboratory test results, demographic information, and clinical measurements data and saves it in a PostgreSQL database;
Step 4a: IPP Coordinator calls preprocessor to process data saved in step 3 and creates a dataset to feed to the model;
Step 4b: IPP Coordinator calls Model startup client class to execute the model using the dataset created in step 4a;
Step 5: The model produces an output data set including probability of death, model coefficients, a categorical classification of numeric probabilities (e.g. high risk or low risk) for each admission;
Step 6: Model output saved in PostgreSQL database;
Step 7: After completion of all new admissions, IPP Coordinator calls CIS API to push model categorical classification into the EMR/CIS.

The following include potential embodiments of the disclosed approach, representative of other examples that are not intended to be limiting in any way:
Embodiment A1: A method comprising training a machine-learning classifier for predicting likelihoods of patients dying a first number of days following inpatient admission, wherein training the machine-learning classifier comprises: generating a training dataset using data for subjects in a cohort of study subjects, wherein each subject in the cohort of subjects had a subject admission at a subject healthcare facility; applying one or more machine learning techniques to the training dataset such that the machine-learning classifier is configured to receive a set of inputs and provide, as output, likelihood of dying the first number of days following inpatient admission; and providing the machine-learning classifier for use in generating patient-specific likelihoods of dying the first number of days following respective inpatient admissions.
Embodiment A2: The method of Embodiment A1, wherein the training dataset comprises, for each subject, one or more of: (A) numerical values based on a set of laboratory tests run on each subject; (B) categorical values based on the set of laboratory test results; (C) historical values; and/or (D) subject physical characteristic values.
Embodiment A3: The method of either Embodiment A1 or A2, wherein the training dataset comprises, for each subject, numerical values based on a set of laboratory tests run on each subject within a second number of days before or after the subject admission.
Embodiment A4: The method of any of Embodiments A1-A3, wherein the training dataset comprises, for each subject, categorical values based on the set of laboratory test results.
Embodiment A5: The method of any of Embodiments A1-A4, wherein the training dataset comprises, for each subject, subject physical characteristic values.
Embodiment A6: The method of any of Embodiments A1-A5, wherein the training dataset comprises, for each subject, historical values.
Embodiment A7: The method of any of Embodiments A1-A6, wherein the numerical values comprise values corresponding to one or more of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and/or (xv) monocytes.
Embodiment A8: The method of any of Embodiments A1-A7, wherein the categorical values comprise one or more values corresponding to one or more of: (i) albumin, (ii) carbon dioxide (CO2), and/or (iii) calcium corrected for albumin.
Embodiment A9: The method of any of Embodiments A1-A8, wherein the historical values comprise a count of emergent admissions in a predetermined timeframe preceding the subject admission of each subject.
Embodiment A10: The method of any of Embodiments A1-A9, wherein the subject physical characteristic values correspond to at least one of body surface area (BSA), body mass index (BMI), height, or weight.
Embodiment A11: The method of any of Embodiments A1-A10, wherein mortality at the first number of days following the subject admission is known for each subject, and wherein the one or more machine learning techniques comprises one or more supervised machine learning techniques.
Embodiment A12: The method of any of Embodiments A1-A11, wherein the one or more machine learning techniques comprises a regression analysis model.
Embodiment A13: The method of any of Embodiments A1-A12, wherein the regression analysis model is based on Least Absolute Shrinkage and Selection Operator (LASSO).
Embodiment A14: The method of any of Embodiments A1-A13, wherein the first number of days is between 30 and 90.
Embodiment A15: The method of any of Embodiments A1-A14, wherein the first number of days is 45.
Embodiment A16: The method of any of Embodiments A1-A15, wherein each subject in the cohort of subjects had a cancer and/or a non-cancerous disease.
Embodiment A17: The method of any of Embodiments A1-A16, wherein each subject in the cohort of subjects had a solid tumor and/or a hematological malignancy.
Embodiment A18: The method of any of Embodiments A1-A17, further comprising using the machine-learning classifier to predict a likelihood of a patient dying the first number of days following an admission of the patient at a healthcare facility.
Embodiment A19: The method of any of Embodiments A1-A18, wherein using the machine-learning model comprises selecting a set of features for predicting the likelihood a non-zero coefficient, wherein the set of features are selected based on predictive coefficients of features.
Embodiment B1: A method comprising training a machine-learning classifier for predicting likelihoods of patients dying a first number of days following inpatient admission, the training the machine-learning classifier comprising: generating a training dataset using data for subjects in a cohort of study subjects, wherein mortality at the first number of days following the subject admission is known for each subject, and wherein each subject in the cohort of subjects had a subject admission at a subject healthcare facility, the training dataset comprising, for each subject, at least one of: (A) numerical values based on a set of laboratory tests run on each subject within a second number of days before or after the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject physical characteristic values corresponding to at least one of body surface area (BSA), body mass index (BMI), height, or weight; applying one or more supervised machine learning techniques to the training dataset such that the machine-learning classifier is configured to receive, as input, numerical values, categorical values, historical values, and one or more demographic or biometric values and provide, as output, likelihood of dying the first number of days following inpatient admission; and providing the classifier for use in generating patient-specific likelihoods of dying the first number of days following respective inpatient admissions.
Embodiment B2: The method of Embodiment B1, wherein the one or more machine learning techniques comprises a regression analysis model.
Embodiment B3: The method of either Embodiment B1 or B2, wherein the regression analysis model is based on Least Absolute Shrinkage and Selection Operator (LASSO).
Embodiment B4: The method of any of Embodiments B1-B3, wherein the first number of days is between 30 and 90.
Embodiment B5: The method of any of Embodiments B1-B4, wherein each subject in the cohort of subjects had a solid tumor and/or a hematological malignancy.
Embodiment B6: The method of any of Embodiments B1-B5, further comprising using the machine-learning classifier to predict a likelihood of dying the first number of days following an admission of the patient at a healthcare facility.
Embodiment B7: The method of any of Embodiments B1-B6, wherein using the machine-learning model comprises selecting a set of features for predicting the likelihood a non-zero coefficient, wherein the set of features are selected based on predictive coefficients of features.
Embodiment C1: A method comprising: receiving, by a first computing device, for a patient admitted to a healthcare facility, health data comprising at least one of: (A) numerical values based on a set of laboratory tests run on the patient, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject physical characteristic values corresponding to at least one of body surface area (BSA), body mass index (BMI), height, or weight; inputting, by the first computing device, the health data or a subset thereof to a machine-learning classifier to generate a likelihood that the patient will die a predetermined number of days following admission to the healthcare facility, the machine learning classifier having been trained, by the first computing device or a second computing device, by: generating a training dataset using data for subjects in a cohort of study subjects, wherein each subject in the cohort of subjects had a subject admission at a subject healthcare facility, the training dataset comprising, for each subject, at least one of: (A) numerical values based on a set of laboratory tests run on each subject following the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes; (B) categorical values based on the set of laboratory test results, the categorical values comprising values corresponding to at least one of: (i) albumin, (ii) carbon dioxide (CO2), and (iii) calcium corrected for albumin; and (C) subject physical characteristic values corresponding to at least one of body surface area (BSA), body mass index (BMI), height, or weight; and applying one or more machine learning techniques to the training dataset to obtain the classifier such that the configured to receive, as input, numerical values, categorical values, historical values, and one or more demographic or biometric values and provide, as output, likelihood of dying the number of days following inpatient admission; and providing, by the first computing device, the likelihood generated by the machine-learning classifier to a healthcare provider for use in care of the patient.
Embodiment C2: The method of Embodiment C1, further comprising selecting a set of features to be input into the machine-learning classifier, wherein the set of features may be selected based on predictive coefficient values for the features.
Embodiment C3: The method of either Embodiment C1 or C2, wherein the health data or the subset thereof comprises a set of features selected based on predictive coefficient values for the features.
Embodiment C4: The method of any of Embodiments C1-C3, wherein mortality at the number of days following the subject admission is known for each subject, and wherein the one or more machine learning techniques comprises one or more supervised machine learning techniques.
Embodiment C5: The method of any of Embodiments C1-C4, wherein the one or more machine learning techniques comprises a LASSO regression model.
Embodiment C6: The method of any of Embodiments C1-C5, wherein the number of days is between 40 and 50.
Embodiment D1: A computing system comprising one or more processors configured to implement any of Embodiments A1-C6.
Embodiment E1: A non-transitory computer-readable storage medium with instructions configured to cause one or more processors of a computing system to implement any of Embodiments A1-C6.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method comprising training a machine-learning classifier for predicting likelihoods of patients dying a first predetermined number of days following inpatient admission, wherein training the machine-learning classifier comprises:
generating a training dataset using data for subjects in a cohort of study subjects, wherein each subject in the cohort of subjects had a subject admission at a subject healthcare facility, the training dataset comprising, for each subject:
(A) numerical values based on a set of laboratory tests run on each subject within a second predetermined number of days before or after the subject admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes;
(B) one or more categorical values based on the set of laboratory test results, the categorical values comprising values corresponding to at least one of: (i) albumin, (ii) carbon dioxide (CO2), or (iii) calcium corrected for albumin;
(C) one or more historical values corresponding to one or more prior admissions; and
(D) subject demographic or biometric values corresponding to at least one of body surface area (BSA), body mass index (BMI), height, or weight;
applying one or more machine learning techniques to the training dataset such that the machine-learning classifier is configured to receive, as input, numerical values, categorical values, historical values, and one or more demographic or biometric values and provide, as output, likelihood of dying the first predetermined number of days following inpatient admission; and
providing the machine-learning classifier for use in generating patient-specific likelihoods of dying the first predetermined number of days following respective inpatient admissions.

2. The method of claim 1, wherein mortality at the first predetermined number of days following the subject admission is known for each subject, and wherein the one or more machine learning techniques comprises one or more supervised machine learning techniques.

3. The method of claim 1, wherein the one or more machine learning techniques comprises a regression analysis model.

4. The method of claim 4, wherein the regression analysis model is based on Least Absolute Shrinkage and Selection Operator (LASSO).

5. The method of claim 1, wherein the first predetermined number of days is between 30 and 90.

6. The method of claim 6, wherein the first predetermined number of days is 45.

7. The method of claim 1, wherein each subject in the cohort of subjects had a cancer.

8. The method of claim 1, further comprising predicting, using the machine-learning classifier, a likelihood of a patient dying the first predetermined number of days following an admission of the patient at a healthcare facility.

9. The method of claim 1, wherein using the machine-learning classifier comprises selecting a set of features for predicting the likelihood a non-zero coefficient, wherein the set of features are selected based on predictive coefficients of features.

10. The method of claim 1, further comprising retraining the machine-learning classifier using a second training dataset comprising data on one or more subjects not included in the original cohort of subjects to obtain ad updated machine-learning classifier.

11. A method comprising training a machine-learning classifier for predicting likelihoods of patients dying a first predetermined number of days following inpatient admission, wherein training the machine-learning classifier comprises:
generating a training dataset using data for subjects in a cohort of study subjects, wherein mortality at the first predetermined number of days following the subject admission is known for each subject, and wherein each subject in the cohort of subjects had a subject admission at a subject healthcare facility, the training dataset comprising, for each subject:
(A) numerical values based on a set of laboratory tests run on each subject within a second predetermined number of days before or after the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes;
(B) categorical values based on the set of laboratory test results, the categorical values comprising values corresponding to at least one of: (i) albumin, (ii) carbon dioxide (CO2), and (iii) calcium corrected for albumin; and
(C) subject demographic or biometric values corresponding to at least one of body surface area (BSA), body mass index (BMI), height, or weight;
applying one or more supervised machine learning techniques to the training dataset such that the machine-learning classifier is configured to receive, as input, numerical values, categorical values, historical values, and one or more demographic or biometric values and provide, as output, likelihood of dying the first predetermined number of days following inpatient admission; and
providing the classifier for use in generating patient-specific likelihoods of dying the first predetermined number of days following respective inpatient admissions.

12. The method of claim 11, wherein the one or more machine learning techniques comprises a regression analysis model.

13. The method of claim 12, wherein the regression analysis model is based on Least Absolute Shrinkage and Selection Operator (LASSO).

14. The method of claim 11, wherein the first predetermined number of days is between 30 and 90.

15. The method of claim 11, wherein each subject in the cohort of subjects had a solid tumor.

16. The method of claim 11, further comprising using the machine-learning classifier to predict a likelihood of dying the first predetermined number of days following an admission of the patient at a healthcare facility.

17. A method comprising:
receiving, by a first computing device, for a patient admitted to a healthcare facility, health data comprising:
(A) numerical values based on a set of laboratory tests run on the patient, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (Xv) monocytes;
(B) categorical values based on the set of laboratory test results, the categorical values comprising values corresponding to at least one of: (i) albumin, (ii) carbon dioxide (CO2), and (iii) calcium corrected for albumin; and
(C) subject demographic or biometric values corresponding to at least one of body surface area (BSA), body mass index (BMI), height, or weight;
inputting, by the first computing device, the health data to a machine-learning classifier to generate a likelihood that the patient will die a predetermined number of days following admission to the healthcare facility, the machine learning classifier having been trained, by the first computing device or a second computing device, by:
generating a training dataset using data for subjects in a cohort of study subjects, wherein each subject in the cohort of subjects had a subject admission at a subject healthcare facility, the training dataset comprising, for each subject:
(A) numerical values based on a set of laboratory tests run on each subject following the admission, the numerical values comprising values corresponding to a plurality of: (i) creatinine, (ii) mean corpuscular volume (MCV), (iii) mean corpuscular hemoglobin (MCH), (iv) anion gap, (v) blood urea nitrogen (BUN), (vi) red blood cell distribution width (RDW), (vii) lymphocytes, (viii) potassium, (ix) alkaline phosphatase (ALK), (x) aspartate aminotransferase (AST), (xi) eosinophils, (xii) alanine aminotransferase (ALT), (xiii) platelets, (xiv) protein total plasma, and (xv) monocytes;
(B) categorical values based on the set of laboratory test results, the categorical values comprising values corresponding to at least one of: (i) albumin, (ii) carbon dioxide (CO2), and (iii) calcium corrected for albumin; and
(C) subject demographic or biometric values corresponding to at least one of body surface area (BSA), body mass index (BMI), height, or weight; and
applying one or more machine learning techniques to the training dataset to obtain the classifier such that the configured to receive, as input, numerical values, categorical values, historical values, and one or more demographic or biometric values and provide, as output, likelihood of dying the predetermined number of days following inpatient admission; and
providing, by the first computing device, the likelihood generated by the machine-learning classifier to a healthcare provider for use in care of the patient.

18. The method of claim 17, wherein mortality at the predetermined number of days following the subject admission is known for each subject, and wherein the one or more machine learning techniques comprises one or more supervised machine learning techniques.

19. The method of claim 17, wherein the one or more machine learning techniques comprises a LASSO regression model.

20. The method of claim 17, wherein the predetermined number of days is between 40 and 50.
